(19) United States
(12) Patent Application Publication
(10) Pub. No.: US 2025/0021330 A1
(43) Pub. Date: Jan. 16, 2025

(54) SAFE SOFTWARE VERSIONING FOR MEDICAL APPLICATION

(71) Applicant: Align Technology, Inc., San Jose, CA (US)

(72) Inventors: Galina Nechaeva, Krasnogorsk (RU); Stanislav Makhrov, Darmstadt (DE); Nikita Skiba, Domodedovo (RU); Anna Azizova, Raleigh, NC (US); Evgeny Koptybenko, Cary, NC (US); Svetlana Mikhalina, Madrid (ES); Daniil Eliseev, Cary, NC (US)

(21) Appl. No.: 18/769,055

(22) Filed: Jul. 10, 2024

Related U.S. Application Data
(60) Provisional application No. 63/526,831, filed on Jul. 14, 2023.

(51) Int. Cl. G06F 8/71; G06F 8/65; G06F 21/12; G16H 10/60

(52) U.S. Cl. CPC G06F 8/71; G06F 8/65; G06F 21/121; G16H 10/60

(57) ABSTRACT

A method includes receiving a command to execute a medical application having a plurality of versions, the plurality of versions comprising at least a first version and a second version, determining whether one or more selection criteria associated with the second version of the medical application are satisfied, and responsive to determining that the one or more selection criteria are satisfied, executing the second version of the medical application.

### RELATED APPLICATIONS

This patent application claims the benefit under 35 U.S.C. § 119(e) of U.S. Provisional Application No. 63/526,831, filed Jul. 14, 2023, all of which are incorporated by reference herein.

### TECHNICAL FIELD

Embodiments of the present disclosure relate to the field of medical software and, in particular, to a system and method for safely testing new versions of medical software and/or deploying the new versions of the medical software.

### BACKGROUND

Medical software such as that used to develop treatment plans for patients or perform treatments on patients is regulated under a strict set of safety regulations. For example, software as a medical device (SaMD) is software that performs one or more medical functions, and is regulated by various agencies. Regulations for medical software may impose constraints on when a version of the software can be used on patients, on who can use the version of the software, and so on. Additionally, when a new version of medical software is generated, it is important to test that new version of the software before rolling it out for general use in case the new version of the software should have problems that could negatively impact patients. Accordingly, traditionally when a new version of medical software is generated that new version of the medical software is installed on a small number of dedicated computing devices that do not have a production version of the medical software installed. This includes first uninstalling the production version of the software from those computing devices and then installing the new version of the medical software. Uninstalling the old version of the medical software and then installing the new version of the medical software can take hours to perform. Additionally, if the new version of the software ends up being problematic, then it must be uninstalled from the computing device before the old version of the software can be reinstalled, which again takes hours to perform. During such time the computing device is unavailable for use on patient cases, and technicians that use the computing device may be idle.

### SUMMARY

In a first example implementation, a method comprises receiving a command to execute a medical application having a plurality of versions, the plurality of versions comprising at least a first version and a second version; determining whether one or more selection criteria associated with the second version of the medical application are satisfied; and responsive to determining that the one or more selection criteria are satisfied, executing the second version of the medical application.

In a second example implementation, a system comprises: a computing device comprising a memory and one or more processors, the computing device configured to: receive a command to execute a medical application having a plurality of versions, the plurality of versions comprising at least a first version and a second version; determine whether one or more selection criteria associated with the second version of the medical application are satisfied; and responsive to determining that the one or more selection criteria are satisfied, execute the second version of the medical application.

In a third example implementation, a non-transitory computer readable medium comprising instructions that, when executed by a processing device, cause the processing device to perform operations comprising: receiving a command to execute a medical application having a plurality of versions, the plurality of versions comprising at least a first version and a second version; determining whether one or more selection criteria associated with the second version of the medical application are satisfied; and responsive to determining that the one or more selection criteria are satisfied, executing the second version of the medical application.

### BRIEF DESCRIPTION OF THE DRAWINGS

Embodiments of the present disclosure are illustrated by way of example, and not by way of limitation, in the figures of the accompanying drawings.

FIG. 1A illustrates one embodiment of a system for executing medical applications, in accordance with an embodiment.

FIG. 1B illustrates one embodiment of a medical application version selection system, in accordance with an embodiment.

FIG. 2A illustrates a flow diagram for a method of selecting a version of a medical application for execution, in accordance with an embodiment.

FIG. 2B illustrates a flow diagram for a method of selecting a version of a medical application for execution, in accordance with an embodiment.

FIG. 3 illustrates a flow diagram for a method of selecting a version of a medical application for execution, in accordance with an embodiment.

FIG. 4 illustrates a flow diagram for a method of selecting a version of a medical application for execution based at least in part on blacklists, in accordance with an embodiment.

FIG. 5 illustrates a flow diagram for a method of determining whether to execute a stable (production) version of a medical application or a test version of the medical application, in accordance with an embodiment.

FIG. 6 illustrates a flow diagram for a method of selecting a version of a medical application for execution based at least in part on region, in accordance with an embodiment.

FIG. 7 illustrates a flow diagram for a method of selecting versions dependent medical applications for execution, in accordance with an embodiment.

FIG. 8 illustrates a flow diagram for a method of setting criteria for determining which version of a medical application to execute, in accordance with an embodiment.

FIG. 9 illustrates a block diagram of an example computing device, in accordance with embodiments of the present disclosure.

### DETAILED DESCRIPTION

Described herein are methods and systems for selecting versions of medical applications and/or other medical software or execution, in accordance with embodiments of the present disclosure. Medical applications and other medical software is subject to various regulations to ensure patient safety, data security, and the effectiveness of healthcare technology. Specific regulations vary based on country or region. In the United States (US), the Food and Drug Administration (FDA) regulates medical software under the Federal Food, Drug and Cosmetic Act (FD&C Act). Software that meets the definition of a medical device is subject to FDA oversight. Depending on the risk level, medical software may require premarket clearance (e.g., 510(k) clearance) or approval (e.g., Pre-market Approval—PMA) before it can be marketed. As new versions of the medical software are generated, each such version may need to undergo rigorous testing before it can be released for general use in the US. Similarly, medical devices regulation (MDR) in the European Union provides regulations for medical devices, including software, within the European Union. Medical software must meet the requirements outlined in the MDR and may need to undergo conformity assessment procedures, such as certification by a notified body, based on the software's classification. Additionally, the International Medical Device Regulators Forum (IMDRF) is a global organization that harmonizes medical device regulations. The IMDF has released guidance documents related to software as a medical device (SaMD), providing recommendations on risk categorization, clinical evaluation, and cybersecurity. A commonality of each of the regulating bodies for medical applications/software, is an imposition of additional rules and regulations for medical applications/software that do not exist for other types of applications/software.

Software versioning is an important aspect of software development and maintenance, including medical software, to ensure traceability, transparency, and quality control. In order to ensure that changes made to medical applications/software as new versions of the medical applications/software are developed are safe and comply with the appropriate regulatory requirements, it is important that developers for medical applications/software to carefully manage the execution of different versions of the medical applications/software. The process for managing changes in medical software should be well-documented and followed consistently. This includes defining the criteria for releasing a new version, documenting the changes made in each version, and implementing a change control process to ensure proper review, approval, and validation of changes. Medical software developers should conduct risk assessments to identify potential risks associated with software changes and new versions. The risk assessment should consider factors such as the impact on patient safety, data integrity, and regulatory compliance. Each software version should undergo appropriate validation and verification processes to ensure that it performs as intended and meets the necessary requirements. This may include testing the software for functionality, performance, security, and compliance with relevant regulations. Once a software version is released, it is important to monitor its performance in real-world settings. Feedback from users, incident reports, and other forms of post-market surveillance help identify any issues or risks associated with a specific version. This information can be used to inform future updates and improvements and to determine which version of the medical software to execute at a given time.

Traditionally, as a new version of a medical application/software is generated, the new version of the medical application/software is installed on a small number of dedicated computing devices that do not have a stable production version of the medical application installed. This includes first uninstalling the production version of the software from those computing devices and then installing the new version of the medical application. Uninstalling the old version of the medical application and then installing the new version of the medical application can take hours to perform. Additionally, if the new version of the software ends up being problematic, then it must be uninstalled from the computing device before the old version of the software can be reinstalled, which again takes hours to perform. During such time the computing device is unavailable for use on patient cases, and technicians that use the computing device may be idle.

In embodiments, multiple versions of a medical application/software are installed on a computing device. Only a single version of the medical application/software is then exposed to a user in embodiments. Accordingly, the user may not know that there are multiple versions of the medical application present on the computing device. Also installed on the computing device is a concierge application, also referred to herein as a launcher. When a user attempts to execute the medical application, instead the concierge application may be executed. The concierge application may then send a command to a version selector to cause the version selector to select one of the available versions of the medical application/software for execution. The version selector may be installed on the computing device or on a different computing device and/or may be provided as a service (e.g., a cloud-based service). The command may be accompanied by data associated with the execution request, which may include, for example, a user account, a region, a patient record identifier, a list of available versions of the application on a computing device on which the medical application is to be executed, and/or other information. The version selector may then determine what versions of the medical application/software are available on the computing device and apply one or more rules (e.g., version selection criteria) to determine which, if any, of the available versions of the medical application/software should be executed. Once a selection of a version of the medical application/software is made, the version selector may notify the concierge application of the selection, which may then cause the selected version of the medical application/software to be executed.

Embodiments enable multiple versions of the medical application/software to be present on the same computing device without risk of an unauthorized user using the wrong version of the medical application, or of the wrong version of the medical application otherwise being executed. If at any time a particular version of the medical application is determined to be problematic (e.g., if a test version, also referred to as a “canary” version, has caused errors or corrupted one or more patient cases), then this information may be used for future decisions of which version of the medical application to execute and may result in the particular version of the application exhibiting problems being prohibited from further execution. Instead, future requests to execute the medical application may result in a different version of the medical application (e.g., an older stable version of the medical application) being executed. Since multiple versions of the medical application are already installed on the computing device, there is no machine down time or user idle time after determination that the latest version of the medical application is problematic (e.g., is unstable). Additionally, in embodiments there is little to no delay between determining that a version of the medical application is problematic and halting that version of the medical application from further execution. Accordingly, embodiments speed up the process of testing new versions of medical applications/software, reduce down time of machines that might result from detection of problematic versions of the medical applications/software, and increase the speed at which remedial actions are performed after detection of problematic versions of the medical applications/software. Additionally, embodiments enable the same computer to be used for multiple versions of a medical application at the same time, where for some users one version of the medical application is launched and for other users another version of the medical application is launched. In contrast, in prior systems a single computer could only be used with a single version of a medical application, and all users of that computer would use that same version of the medical application.

Embodiments are discussed with reference to medical applications. It should be understood that such embodiments also pertain to medical software other than medical applications. Accordingly, embodiments discussed herein apply to all types of medical software.

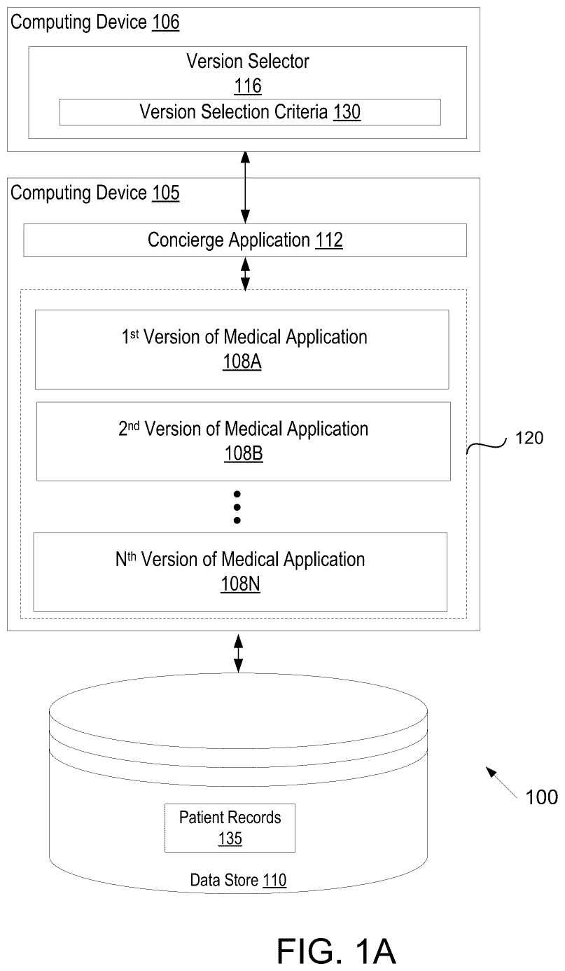

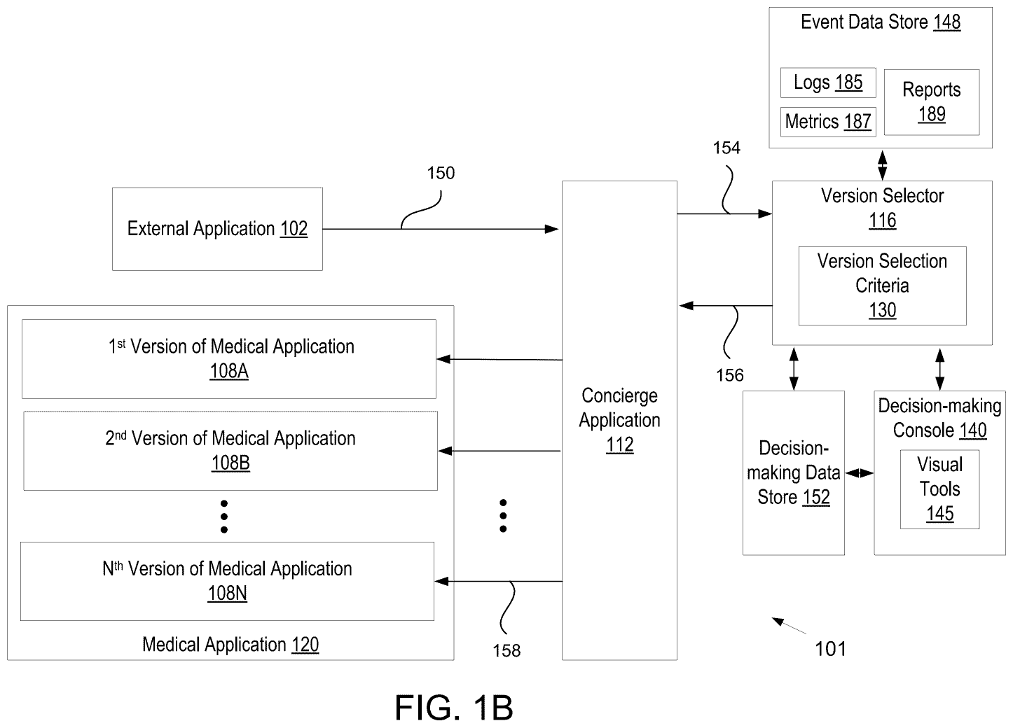

FIG. 1A illustrates one embodiment of a system 100 for executing medical applications, in accordance with an embodiment. In one embodiment, the system 100 includes a computing device 105, a data store 110, and a computing device 106. The computing device 105, 106 may include physical machines and/or virtual machines hosted by physical machines. The physical machines may be rackmount servers, desktop computers, or other computing devices. The physical machines may include a processing device, memory, secondary storage, one or more input devices (e.g., such as a keyboard, mouse, tablet, speakers, or the like), one or more output devices (e.g., a display, a printer, etc.), and/or other hardware components. In one embodiment, the computing device 105, 106 includes one or more virtual machines, which may be managed and provided by a cloud provider system. Each virtual machine offered by a cloud service provider may be hosted on one or more physical machine. Computing device 105, 106 may be connected to data store 110 either directly or via a network. The network may be a local area network (LAN), a public wide area network (WAN) (e.g., the Internet), a private WAN (e.g., an intranet), or a combination thereof.

Data store 110 may be an internal data store, or an external data store that is connected to computing device 105 directly or via a network. Examples of network data stores include a storage area network (SAN), a network attached storage (NAS), and a storage service provided by a cloud provider system. Data store 110 may include one or more file systems, one or more databases, and/or other data storage arrangement.

In some embodiments, the system 100 may additionally include, or take advantage of, a display (not shown), one or more local area networks (LANs) (not shown), and/or a wide area network (WAN) (not shown). Via the LAN, the computing device 105 may connect to a wide area network (WAN) (now shown), and through the WAN to one or more remote computing devices, such as computing device 106. The LAN may include a router, switch, bridge and/or other network device (not shown) that enables communication between multiple devices (e.g., computing device 105 and other devices) connected to the LAN. The network device may provide wired connections to the LAN using, for example, Ethernet ports, universal serial bus (USB) ports and/or Firewire® ports. The network device may additionally provide wireless connections to the LAN using, for example, a Wi-Fi transceiver. The WAN may include a public WAN (e.g., the Internet), a private WAN (e.g., an intranet), or a combination thereof.

In embodiments, computing device 105 includes a medical application 120. Multiple versions 108A, 108B, through 108N of the medical application 120 may be installed on the computing device 105. However, the multiple versions 108A-N of the medical application 120 may not be exposed to a user of the computing device 105. For example, a launcher, file explorer, file system, etc. of the computing device 105 may show only a single medical application 120, without showing that multiple versions of that medical application exist. Furthermore, specific versions 108A-N of the medical application may not be executable by a user or application of the computing device 105. Instead, a user or application may attempt to execute the medical application 120, responsive to which a concierge application 112 and/or version selector 116 may determine which version 108A-N of the medical application 120 to execute or launch and may launch the determined version 108A-N of the medical application 120. The determined version of the medical application 120 may execute and may open one or more specified patient records 135 (also referred to as patient cases). In embodiments, the patient records may include medical images (e.g., x-ray images, panoramic x-ray images, ultrasound images, CBCT scans, intraoral scans, 3D models of patient anatomy (e.g., of patient upper and lower dental arches), patient case details (e.g., patient age, gender, medical conditions, doctor notes and/or analysis), and so on.

In one embodiment, medical application 120 is a dental and/or orthodontic treatment planning application. Medical application 120 may perform treatment planning, for example, for restorative dentistry and/or for orthodontics in some embodiments. The treatment planning application may be responsible for generating a treatment plan that includes a treatment outcome for a patient. The treatment plan may include and/or be based on an initial 2D and/or 3D image of a patient's dental arches. For example, the treatment planning application may receive 3D intraoral images of the patient's dental arches, and may stitch the 3D images together to create a virtual 3D model of the dental arches. Alternatively, the treatment planning application may receive a virtual 3D model of a patient's dental arches. In embodiments, the treatment planning application receives a patient record 135 for a patient case, which may include, for example, intraoral scans, medical images, patient case details, 3D models of the patient's upper and/or lower dental arches, and so on.

The treatment planning application may then determine current positions and orientations of the patient's teeth from the virtual 3D model in the patient record 135 and determine target final positions and orientations for the patient's teeth represented as a treatment outcome. The treatment planning application may additionally or alternatively determine one or more dental prosthetics to be used for a patient, such as a bridge, cap, crown, and so on.

With respect to orthodontic treatment, the treatment planning application may generate a virtual 3D model showing the patient's dental arches at the end of treatment as well as one or more virtual 3D models showing the patient's dental arches at various intermediate stages of treatment. Alternatively, or additionally, the treatment planning application may generate one or more 3D images and/or 2D images showing the patient's dental arches at various stages of treatment. The 3D models for any of the steps of treatment may be manipulated using a medical computer aided drafting (CAD) application in embodiments.

By way of non-limiting example, a dental treatment outcome may be the result of a variety of dental procedures. Such dental procedures may be broadly divided into prosthodontic (restorative) and orthodontic procedures, and then further subdivided into specific forms of these procedures. Additionally, dental procedures may include identification and treatment of gum disease, sleep apnea, and intraoral conditions. The term prosthodontic procedure refers, inter alia, to any procedure involving the oral cavity and directed to the design, manufacture or installation of a dental prosthesis at a dental site within the oral cavity, or a real or virtual model thereof, or directed to the design and preparation of the dental site to receive such a prosthesis. A prosthesis may include any restoration such as implants, crowns, veneers, inlays, onlays, and bridges, for example, and any other artificial partial or complete denture. The term orthodontic procedure refers, inter alia, to any procedure involving the oral cavity and directed to the design, manufacture or installation of orthodontic elements at a dental site within the oral cavity, or a real or virtual model thereof, or directed to the design and preparation of the dental site to receive such orthodontic elements. These elements may be appliances including but not limited to brackets and wires, retainers, clear aligners, or functional appliances. Any of treatment outcomes or updates to treatment outcomes described herein may be based on these orthodontic and/or dental procedures. Examples of orthodontic treatments are treatments that reposition the teeth, treatments such as mandibular advancement that manipulate the lower jaw, treatments such as palatal expansion that widen the upper and/or lower palate, and so on. For example, an update to a treatment outcome may be generated by interaction with a user to perform one or more procedures to one or more portions of a patient's dental arch or mouth.

A treatment plan for producing a particular treatment outcome may be generated by first generating an intraoral scan of a patient's oral cavity. From the intraoral scan a virtual 3D model of the upper and/or lower dental arches of the patient may be generated. A dental practitioner may then determine a desired final position and orientation for the patient's teeth on the upper and lower dental arches, for the patient's bite, and so on. This information may be used by the treatment planning application to generate a virtual 3D model of the patient's upper and/or lower arches after orthodontic treatment. This data may be used to create an orthodontic treatment plan. The orthodontic treatment plan may include a sequence of orthodontic treatment stages. Each orthodontic treatment stage may adjust the patient's dentition by a prescribed amount, and may be associated with a 3D model of the patient's dental arch that shows the patient's dentition at that treatment stage.

In some embodiments, the treatment planning application may receive or generate one or more virtual 3D models, virtual 2D models, 3D images, 2D images, or other treatment outcome models and/or images, which may be based on intraoral images or scans. For example, an intraoral scan of the patient's oral cavity may have been performed to generate an initial virtual 3D model of the upper and/or lower dental arches of the patient. The treatment planning application may then determine a final treatment outcome based on the initial virtual 3D model, and then generate a new virtual 3D model representing the final treatment outcome. The treatment planning application may additionally determine various intermediate stages of orthodontic treatment, and generate virtual 3D models of the patient's dental arches for each such intermediate stage. Clinically important factors such as an amount of force to be applied to teeth, an amount of rotation to be achieved by teeth, an amount of movement of teeth, teeth interactions, and so on should be considered by the treatment planning application in generation of the intermediate treatment stages.

Once a treatment plan is finalized, the various 3D models of the patient's dental arches for each of the stages of treatment may be used to manufacture orthodontic aligners for each of the stages of treatment. The patient may then wear the orthodontic aligners in order for treatment. At the end of treatment, the patient should have corrected dentition.

Different versions 108A-N of the medical application 120 may process the same patient records 135, but yield different results. Generally, at any given time there will be at least one stable or production version of the medical application 120. The stable version has undergone rigorous testing and is known to produce clinically acceptable results. As improvements are made to the medical application 120, new versions of the medical application may be developed. However, until such new versions of the medical application 120 are thoroughly tested, it may be unknown whether those versions of the medical application 120 will yield clinically acceptable results, whether they will be stable, whether they might introduce errors, whether they might corrupt patient cases, and so on. Accordingly, new versions of the medical application 120 should generally be tested carefully before being released for general use.

In embodiments, when an attempt is made to launch or execute medical application 120 (e.g., by a user or by an external application), concierge application 112 is instead executed. The attempt to launch or execute the medical application 120 may include information such as a user account used to attempt to execute the medical application 120, a region (e.g., geographic region or other designated region) associated with the computing device, a patient record 135 to be accessed, and so on. Concierge application 112 may invoke version selector 116 with a request for the version selector 116 (e.g., via an application programming interface (API) of the version selector 116) to select aversion of the medical application 120 to execute. In the illustrated example, version selector 116 is an application that runs on computing device 105. Alternatively, version selector 116 may execute on computing device 106. In one embodiment, version selector 116 is a service (e.g., a cloud-base service) that concierge application 112 may make a call to.

In embodiments, version selector 116 executes on a separate computing device 106 (e.g., on a remote server) than computing device 105. The computing device (e.g., remote server) 106 may receive version selection requests from multiple different computing devices, and may make version selections and provide version selection recommendations to concierge applications 112 running on the multiple different computing devices. Such a version selector 116 may include up-to-date rules or criteria that govern the selection of versions of the medical application, may have access to error logs and/or records of the various versions of the medical application, and/or may manage which versions of the medical application 120 are used across a region and/or the globe in embodiments.

Version selector 116 applies one or more rules to determine which version of the medical application 120 should be launched. The rules may be or include one or more version selection criteria 130. Version selector 116 may receive information from concierge application and/or other sources, and use the received information to make an informed decision as to which version of the medical application 120 should be executed. For example, the version selector 116 may consider a user account, region, patient record, number of instances of various versions of the medical application being executed, number of patient cases being handled under the various versions of the medical application, historical information about execution of the various versions of the medical application and errors associated with such execution (e.g., from a data store or database with monitoring, logging and/or metrics on the various versions of the medical application), and so on. Version selector 116 may determine an appropriate version 108A-N of the medical application to execute, or may determine that no version of the medical application 120 should be executed, and may return a recommendation to execute the determined version to concierge application 112. Concierge application 112 may then cause the recommended version 108A-N of the medical application 120 to be executed.

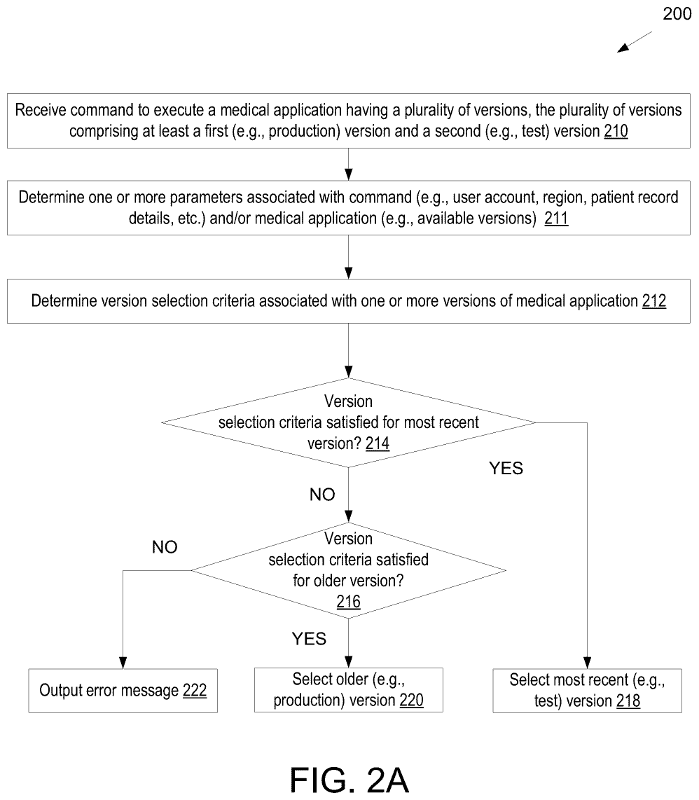

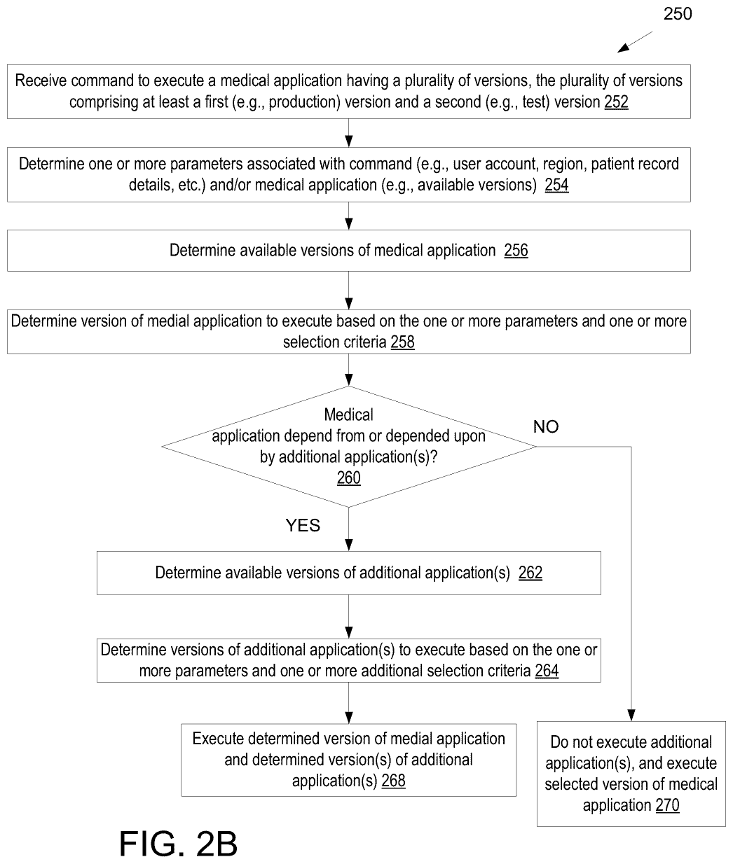

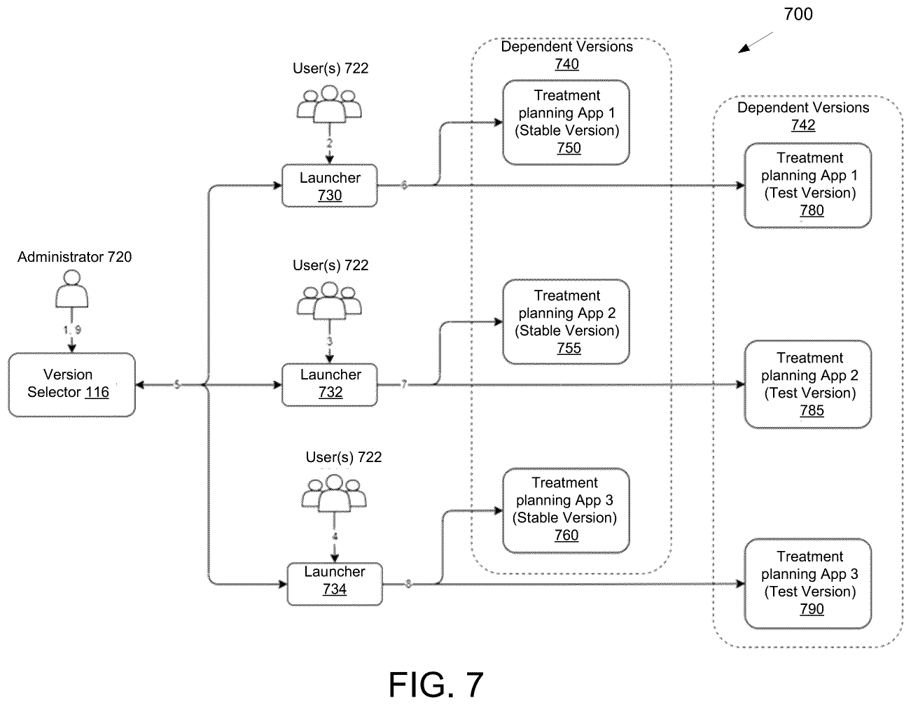

While FIG. 1A shows selection and execution of a version of a single medical application, the same techniques described with reference to a single medical application also apply for selection and execution of chains of interrelated medical applications. For example, often multiple different related medical applications will operate together. In order to perform treatment planning, for example, a treatment planning computer aided drafting (CAD) application may be used, a treatment plan generation application may be used, a treatment plan calculation application may be used, and so on. Each of these treatment plan-related applications may be dependent on one or more other treatment plan-related applications. Some versions of one medical application may be compatible with a first version of another medical application on which it depends, but may not be compatible with a second version of the other medical application, for example. Accordingly, it can be important to ensure that the proper versions of a chain or collection of interrelated and/or interdependent medical applications are executed for proper functioning. In embodiments, a different concierge application and/or version selector 116 may be used for each of the different interrelated medical applications. Each such version selector 116 may include its own set of version selection criteria 130. Alternatively, a single concierge application 112 and/or version selector 116 may operate for multiple different medical applications. In such an instance, the single medical selector 116, for example, may include different rule sets (e.g., different version selection criteria 130) for different medical applications. The selection of interdependent medical applications is described in greater detail below with reference to FIGS. 2B and 7.

FIG. 1B illustrates one embodiment of a medical application version selection system 101, in accordance with an embodiment. As shown, an external application 102 or a user may attempt to execute the medical application 120, which may cause execution 150 of concierge application 112. Concierge application 112 may then send a version selection request 154 to version selector 116. The version selection request 154 may include information such as, for example, a user account, a patient record, a list of available versions of the medical application, and so on. Additionally, version selector 116 may access an event data store 148 to obtain information (e.g., which may contain logs 150, metrics 187, reports 189, etc.) associated with various versions of the medical application. Version selector 116 may additionally access decision-making data store 152 to receive information on which user accounts, medical records, etc. have been associated with which versions of the medical application in the past (e.g., which versions of the medical application have previously opened the medical record). For example, the decision making data store 152 may provide information of which medical records were previously processed or opened using a particular version of the medical application. Version selector 116 at any time may additionally receive updates to its version selection criteria 130 from a decision-making console 140 or other source, and may update the version selection criteria 130 accordingly. Based on the information at hand and the version selection criteria, version selector may determine which version selection criteria of which versions of the medical application are satisfied. Based on which version selection criteria 130 of which version of the medical application are satisfied, version selector 116 may make a selection of a version 108A-N of the medical application 120.

Version selector 116 provides a recommendation 156 of a version 108A-N of the medical application 120 to select to concierge application 112. Concierge application 112 then invokes (e.g., executes or launches) 158 the appropriate version of 108A-N of the medical application 120 (or executes no version of the medical application if the recommendation from version selector 116 was not to execute any version of the medical application 120).

In embodiments, a decision-making console 140 may be provided for access by an administrator (e.g., that is responsible for development of the medical application 120). Decision-making console 140 may provide one or more interface for updating the selection criteria 130 and/or generating new selection criteria 130. The interface may include a graphical user interface, a functional interface, a command-line interface, a menu-driven interface, a natural language interface, a touch-based interface, a gesture-based interface, a voice user interface, and so on. A graphical user interface (GUI) utilizes visual elements such as windows, icons, buttons, menus, and text fields. Users interact with the software by clicking, dragging, typing, and selecting options presented on the screen. GUIs are typically designed to be intuitive and user-friendly, allowing users to navigate and perform tasks visually. A command-line interface (CLI) requires users to enter text commands in a specific syntax to interact with the software. Users communicate with the software by typing commands and parameters, and the software responds with textual feedback. Menu-driven interfaces present users with a set of options organized in menus and submenus. Users navigate through the menus using arrow keys, mouse clicks, or touch interactions to make selections. Natural language interfaces allow users to interact with software using spoken or written language. Users can ask questions or issue commands in their natural language, and the software processes and interprets the input to provide appropriate responses or perform desired actions.

Decision-making console 140 may provide one or more tools that enable a user to generate version selection criteria without performing programming (e.g., without knowing the specifics of a programming language). The decision-making console 140 may receive rules in the form of natural language, a sequence of blocks, or other form that does not comply with a programming language syntax. The decision-making console 140 may then translate the instructions into a programming language. The decision-making console 140 may then provide to version selector 130 one or more updates to the version selection criteria based on the translated instructions. In one embodiment, the version selection criteria 130 are implemented in a scripting language. In one embodiment, the version selection criteria 130 can be updated without reprogramming or redeploying the version selector 116, any version of the medical application 120, or the concierge application 112. Instead, one or more configuration files may be generated that contain version selection criteria, which may be provided to version selector 116. Alternatively, one or more existing configuration files may be updated in view of updated version selection criteria 130.

In some embodiments, decision-making console 140 includes a set of visual tools 145 that enable a user to graphically configure rules or criteria for version selection. The version selection tools may include, for example, graphical blocks associated with variables, dependencies, constructors, and so on. A user may select appropriate types of blocks (e.g., which may be color coded), and arrange them in a manner to form a rule. Such selection and arrangement may be performed via, for example, standard drag and drop instructions of a GUI.

In embodiments, decision-making console 140 provides a visual rules editor that allows users to program version selection criteria using blocks of rule components that can be snapped together. The decision-making console 140 presents users with a workspace where they can drag and drop blocks from a palette onto a workspace canvas. Each block represents a specific concept or function associated with a version selection criterion. Blocks may be categorized into groups based on their functionality, such as control flow, math operations, variables, and more. Users can snap blocks together to create a sequence of instructions for a rule. The blocks are designed to fit together logically, ensuring that only compatible blocks can be connected. Blocks have connectors or “plugs” that allow them to be connected to other blocks, creating a visual representation of the version selection criterion's structure. As users connect blocks together, decision-making console 140 may generate the corresponding code in a specific programming language. In embodiments, decision-making console 140 supports multiple programming languages, including Java, JavaScript, Python, Lua, and more.

FIGS. 2A-8 below describe methods of selecting which versions of medical applications to execute, in accordance with embodiments of the present disclosure. The methods depicted in FIGS. 2A-8 may be performed by a processing logic that may comprise hardware (e.g., circuitry, dedicated logic, programmable logic, microcode, etc.), software (e.g., instructions run on a processing device), or a combination thereof. Various embodiments may be performed by a computing device 105 and/or computing device 106 as described with reference to FIG. 1A. In embodiments, some operations are performed on computing device 105 and some operations are performed on computing device 106.

FIG. 2A illustrates a flow diagram for a method 200 of selecting a version of a medical application for execution, in accordance with an embodiment. At block 210 of method 200, processing logic receives a command to execute a medical application having a plurality of versions. In an example, the plurality of versions include at least a first (e.g., production or stable) version and a second (e.g., test) version. The test version may be a new version that has not undergone sufficient testing to be approved for general release yet.

At block 211, processing logic determines one or more parameters associated with the command to execute the medical application and/or associated with the medical application. For example, the command may have originated from a particular user, and may be associated with a user account of that user. In another example, processing logic may determine a region associated with the command's source. Processing logic may additionally or alternatively determine which versions of the medical application are available. Processing logic may additionally or alternatively determine a patient record to be operated on or opened by the medical application or otherwise associated with the medical application. In some embodiments, the command includes a list of available versions of the medical application that are installed on a computing device from which the command originated. One or more of the determined parameters may have been included in the command to execute the medical application in embodiments.

At block 212, processing logic may determine one or more version selection criteria to apply. There may be different version selection criteria associated with each of the versions of the medical application. In some embodiments, there is a set of version selection criteria that dictate which version of the medical application to select. In some embodiments, only a limited number of users are permitted to use the test version of the medical application. One selection criterion may be a rule to select the test version of the medical application only for a limited number of user accounts on a whitelist. Processing logic may determine whether a region associated with the computing device on which processing logic executes (e.g., a region associated with the medical application installed on the computing device) is allowed to be run in the region. Processing logic may additionally or alternatively determine whether a patient record to be operated on by the medical application or otherwise associated with the medical application satisfies one or more criteria for the test version or the stable version of the medical application. Processing logic may additionally or alternatively determine whether the patient record was previously operated on or otherwise associated with a particular version of the medical application and determine which version of the application to use for operation on the patient record based on such historical use information. Other rules and criteria may also be applied for version selection.

At block 214, processing logic determines whether one or more version selection criteria have been satisfied for selection of the most recent version of the medical application (e.g., for the test version). If all version selection criteria associated with the most recent version of the medical application have been satisfied (e.g., a user account is on a white list for use of the latest version of the medical application, a current region is a region selected for testing of the latest version of the medical application, etc.), then the method proceeds to block 218 and the most recent (e.g., test) version of the medical application is selected for use. If not all of the applicable selection criteria are satisfied for the most recent version of the medical application, then the method continues to block 216.

In one embodiment, processing logic determines whether the second or newest version of the medical application is on a blacklist. An execution criterion of the one or more selection criteria may be satisfied by the second version of the medical application not being on the blacklist.

In one embodiment, processing logic determines whether a user account associated with the command to execute the medical application is authorized to execute the second or newest version of the medical application. An execution criterion of the one or more selection criteria may be satisfied by the user account being authorized to execute the second version of the medical application.

In one embodiment, processing logic determines whether the second or newest version of the medical application authorized for use in the current region. An execution criterion of the one or more selection criteria may be satisfied by the second version of the medical application being authorized for use in the current region.

In one embodiment, processing logic determines a number of instances of the second version of the medical application being run. Processing logic may determine whether the number of instances of the second or newest version of the medical application is below a threshold. An execution criterion of the one or more selection criteria may be satisfied by the number of instances of the second version of the medical application being below the threshold.

In one embodiment, an identified patient record is to be accessed or opened by the medical application. Processing logic may determine whether the patient record was previously accessed using any version of the medical application. A criterion of the one or more criteria may be satisfied base on at least one of a) the patient record having been previously accessed by the second version of the medical application or b) the patient record not having been accessed by any other version of the medical application that has a higher version number than the second version of the medical application.

Logs may be kept of problems associated with the newest or test version of the medical application (and optionally of other versions of the medical application). Additionally, or alternatively, a report of the problems associated with the newest version and/or other versions of the medical application may be available, where the report may indicate a number and/or types of problems associated with the newest (and/or other) version(s) of the medical application. Processing logic may determine a number of problems associated with the second or newest version of the medical application (e.g., from the logs and/or report). Processing logic may then determine whether the number of problems associated with the second version of the medical application is below a threshold. An execution criterion of the one or more selection criteria may be satisfied by the number of problems associated with the second version of the medical application being below the threshold.

Some versions of the medical application may be more suited for particular types of patient cases. For example, a newest version of the medical application may include new capabilities to treat new types of clinical issues (e.g., new types or classes of malocclusion) that are not treatable under older versions of the medical application. In another example, some types of clinical conditions of a patient may be known to be difficult to treat, and it may be better to rely on a proven stable version of the medical application for treatment of such clinical conditions. In one embodiment, processing logic determines a patient record to be accessed by the medical application, determines one or more details of the patient record, and determines which of the plurality of versions of the medical application to execute based at least in part on the one or more details of the patient record.

Some doctors may have preferences for using the latest version of the medical application or for using the most stable version of the medical application. A doctor identity may be included in patient case details (e.g., in a patient record), and may be used to help determine which version of the medical application to select.

Any one or more of the above criteria and/or other criteria may be used to determine which version of the medical application to select.

At block 216, processing logic determines whether version selection criteria are satisfied for one or more older version (e.g., a stable version) of the medical application. If the selection criteria are also not satisfied for an older version of the medical application, then no version of the medical application may be selected, and at block 222 processing logic may output an error message (e.g., a message to halt execution). If at block 216 processing logic determines that the selection criteria for an older version of the medical application are satisfied (e.g., the user account has permission to use the older version of the application, and a different or newer version of the medical application hasn't been previously used to process the patient record), then the method may continue to block 220 and the older (e.g., stable or production) version of the medical application may be selected.

FIG. 2B illustrates a flow diagram for a method 250 of selecting a version of a medical application for execution, in accordance with an embodiment. At block 252 of method 250, processing logic receives a command to execute a medical application having a plurality of versions. In an example, the plurality of versions include at least a first (e.g., production or stable) version and a second (e.g., test) version. The test version may be a new version that has not undergone sufficient testing to be approved for general release yet.

At block 254, processing logic determines one or more parameters associated with the command to execute the medical application and/or associated with the medical application. For example, the command may have originated from a particular user, and may be associated with a user account of that user. In another example, processing logic may determine a region associated with a computing device on which processing logic executes. Processing logic may additionally or alternatively determine which versions of the medical application are available. Processing logic may additionally or alternatively determine a patient record to be operated on by the medical application or otherwise associated with the medical application. One or more of the determined parameters may have been included in the command to execute the medical application in embodiments.

At block 256, processing logic may determine which versions of the medical application are available to choose from. In some embodiments, a list of available versions of the medical application are included in the command. In some embodiments, a list of available versions of the medical application is determinable by processing logic (e.g., based on a lookup operation).

At block 258, processing logic determines a version of the medical application to execute based on the one or more parameters and the one or more selection criteria. The parameters may include, for example, user account, doctor identity, patient case details, whether a patient case was previously processed by the medical application, which version of the medical application previously processed the medical application, a current region, number of instances of one or more versions of the medical application in use, error logs and/or reports, blacklists, whitelists, and so on.

At block 260, processing logic determines whether the medical application depends from or is depended upon by one or more additional applications (e.g., additional medical applications). If the medical application is not interdependent with any other application, the method proceeds to block 270. At block 270, the selected version of the medical application is executed, but no additional applications are executed. If the medical application is interdependent with one or more other applications (e.g., one or more additional medical applications), then the method continues to block 262.

At block 262, processing logic determines which versions of the additional applications are available. At block 264, processing logic determines, for each of the other applications (e.g., other medical applications) that depends on the medical application or that is depended upon by the medical application, an appropriate version of that additional operation to select. The selection process for each of the other applications may be the same selection process as previously described for a single medical application. For such additional applications, one selection criterion may be based on which version of the initial medical application was selected. For example, there may be a test version and a stable version of each of two, three or more interrelated and/or interdependent medical applications. In an embodiment, if a stable version is selected for any of the medical applications, then the stable version is selected for each of the other medical applications as well. In one embodiment, if a test version is selected for any of the medical applications, then the test version is selected for each of the other medical applications as well. In some instances, one version of one medical application may be compatible with multiple versions of another medical application. Accordingly, if a first version of a first medical application is selected, then this may narrow selection of a version of a second medical application to a list of available versions of the second medical application that are compatible with the selected version of the first medical application.

At block 284, processing logic executes the determined version of the medical application and additionally executes the determined versions of the other applications, respectively.

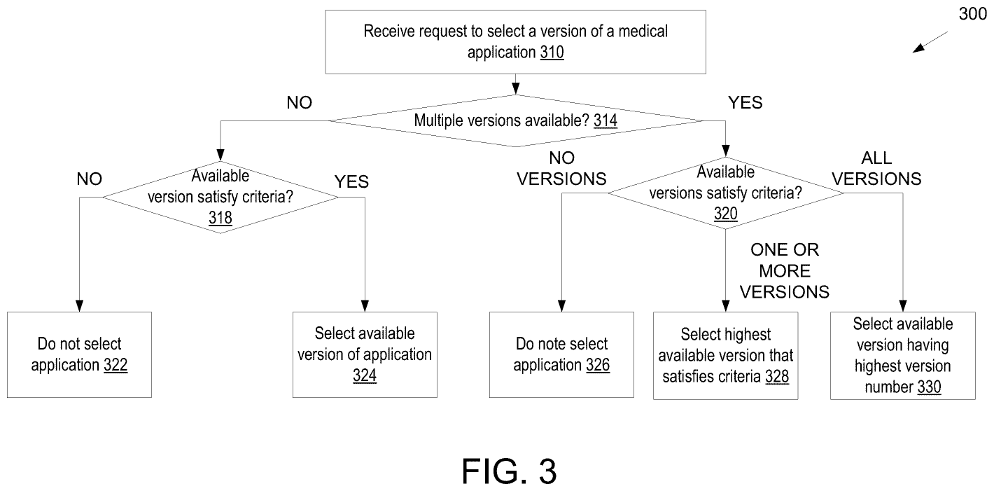

FIG. 3 illustrates a flow diagram for a method 300 of selecting a version of a medical application for execution, in accordance with an embodiment. At block 310 of method 300, processing logic receives a request to select a version of a medical application. At block 314, processing logic determines if multiple versions of the medical application are available. If multiple versions are available, the method proceeds to block 320. If only a single version is available, the method continues to block 318.

At block 318, processing logic determines if the single available version of the medical application satisfies selection criteria. For example, processing logic may determine whether the single version of the medical application is on a blacklist, or if a user account associated with the command is on a blacklist for the available version of the medical application. If the single available version of the medical application satisfies the selection criteria, the method continues to block 324 and the single available version of the medical application is selected. If the single available version of the medical application does not satisfy the selection criteria, then the method continues to block 322 and no version of the medical application is selected or executed.

At block 320, processing logic determines whether one or more available versions of the medical application satisfy the selection criteria. For example, processing logic may determine whether any of the available versions of the medical application is on a blacklist, or if a user account associated with the command is on a blacklist for the available version of the medical application. For example, a blacklist may be implemented in which old and unused versions of the medical application are added to the blacklist as they become outdated to ensure that the old versions of the medical application are not used. If no versions of the medical application satisfy the selection criteria, the method continues to block 326 and no version of the medical application is selected for execution. If at block 320 processing logic determines that one or a few available versions of the medical application satisfy the selection criteria, then at block 328 processing logic selects a highest available version that satisfies the selection criteria for execution. If at block 320 processing logic determines that all available versions of the medical application satisfy the selection criteria, then processing logic selects a highest available version of the medical application for execution.

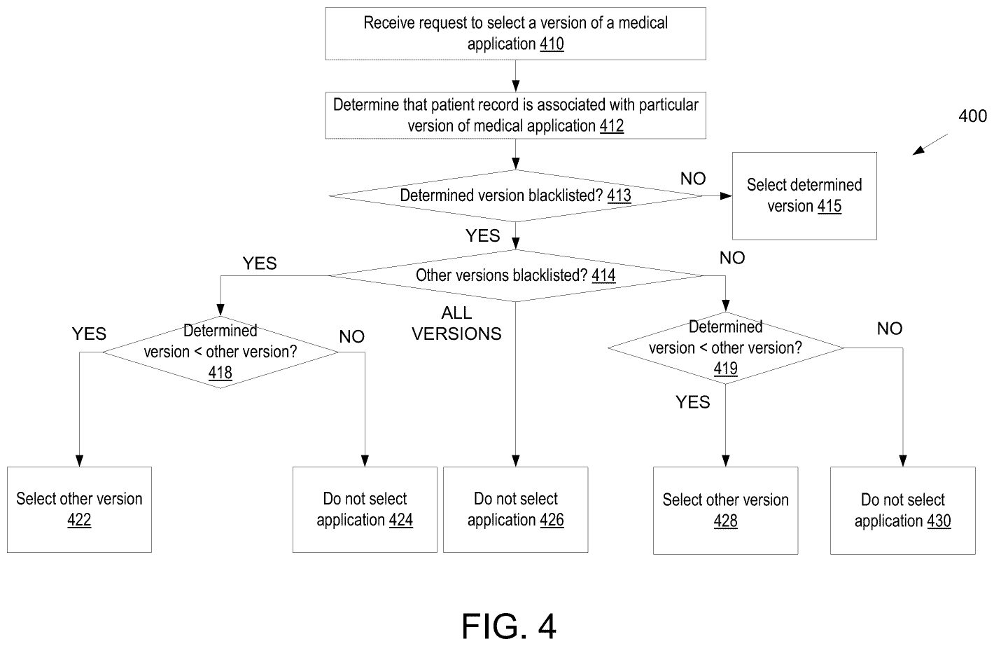

FIG. 4 illustrates a flow diagram for a method 400 of selecting a version of a medical application for execution based at least in part on blacklists, in accordance with an embodiment. Once a treatment plan has been started or generated for a patient case by a version of the medical application, it may be best to continue to access, update and/or complete the treatment plan for that patient case using the same version of the medical application or a newer version of the medical application. Changing which version of the medical application is accessing and/or manipulating the treatment plan for the patient case may cause unforeseen consequences, may corrupt the patient records and/or treatment plan, and/or may cause other negative side effect in some instances. For example, new features, new treatment options, etc. may be added in new versions of the medical application that were not available in older versions of the medical application. Accordingly, if a medical record has previously been opened by a version of the medical application, then it may not be recommended to later open that medical record using an older version of the medical application that may lack a capability to handle some features that may have already been added to a treatment plan for the medical record on a previous opening of the medical record. Accordingly, a record may be kept of which versions of the medical application have previously accessed which patient records. Additionally, a blacklist of versions of the medical application may also be maintained, and may be used to discontinue older versions of the medical application that have become obsolete and make sure that those obsolete versions of the medical application are not used. Additionally, instabilities may be identified in some versions of the medical application, and as a result those versions of the medical application may be blacklisted.

At block 410 of method 400, processing logic may receive a request to select a version of the medical application. The request may be associated with a patient record to be accessed. At block 412, processing logic determines that the patient record is associated with a particular version of the medical application (e.g., that the particular version of the medical application was previously used to access the patient record). This may be determined by performing a lookup into a data store or database using the patient record as a key in one embodiment. The data store may maintain a record of accesses of patient records by versions of the medical application.

At block 413, processing logic determines whether the determined version of the medical application is on a version blacklist. If the determined version of the medical application is not on a blacklist, then the method continues to block 415 and the determined version of the medical application is selected for execution. If the determined version of the medical application is on a blacklist, the method continues to block 414.

At block 414, processing logic determines whether the any other available versions of the medical application are on the version blacklist. If other versions of the medical application are on the blacklist and there are other available versions of the medical application that are not on the blacklist, the method continues to block 418. If all available versions of the medical application are on the blacklist, the method continues to block 426 and no version of the medical application is selected for execution. If one or more versions of the medical application is not on the blacklist, the method continues to block 419.

At block 418, processing logic determines whether the determined version of the medical application is lower than or equal to version numbers of other available versions of the medical application that are not blacklisted. If the determined version of the medical application is lower than or equal to one such other version of the medical application (e.g., there is a newer version of the medical application than the selected version of the application that is not blacklisted), then at block 422 that other version of the medical application is selected for execution. If there is no newer version of the medical application that is available and not blacklisted, then no version of the medical application may be selected at block 424.

At block 419, processing logic determines whether the determined version of the medical application has a lower version number than another available version of the medical application. If the determined application does have a lower version number than another available version of the medical application, the method continues to block 428 and that other version of the medical application having the higher version number is selected. If the determined application does not have a lower version number than the other version of the medical application, then the method continues to block 430 and no version of the medical application is selected for execution.

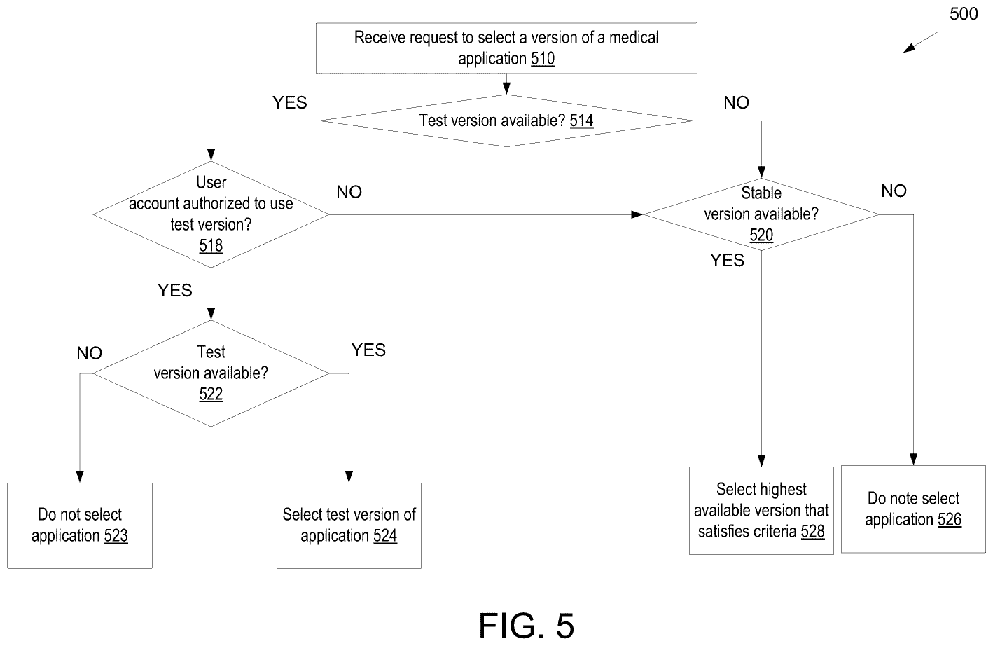

FIG. 5 illustrates a flow diagram for a method 500 of determining whether to execute a stable (production) version of a medical application or a test version of the medical application, in accordance with an embodiment. In embodiments, a test version (canary version) of a medical application may be introduced. A limited number of users may be qualified to use the test version of the medical application. Accordingly, a whitelist may be implemented, in which only user accounts in the white list are authorized to execute the test version of the medical application.

At block 510 of method 500, processing logic receives a request to select a version of a medical application. At block 514, processing logic determines whether a test version of the medical application is available. If a test version is available, the method continues to block 518. If no test version is available, the method continues to block 520.

At block 518, processing logic determines whether a current user (e.g., whether a user account associated with the request) is authorized to use the test version of the medical application. This may include checking whether the user account is on a whitelist associated with the test version of the medical application. If the user account is not authorized to use the test version, the method proceeds to block 520. If the user account is authorized to use the test version, the method continues to block 522.

At block 522, processing logic determines whether the test version of the medical application is available on the computing device on which the medical application will be executed. If the test version is available, the method continues to block 524 and the test version of the medical application is selected for execution. If the test version is not available, then no version of the medical application may be selected for execution (e.g., a halt command or recommendation may be output).

At block 520, processing logic determines whether one or more stable or production versions of the medical application is available on the computing device on which the medical application is to be executed. If one or more stable versions of the medical application are available, the method continues to block 528 and a highest available stable version of the medical application is selected for execution. If no stable version of the medical application is available, then no version of the medical application may be selected for execution.

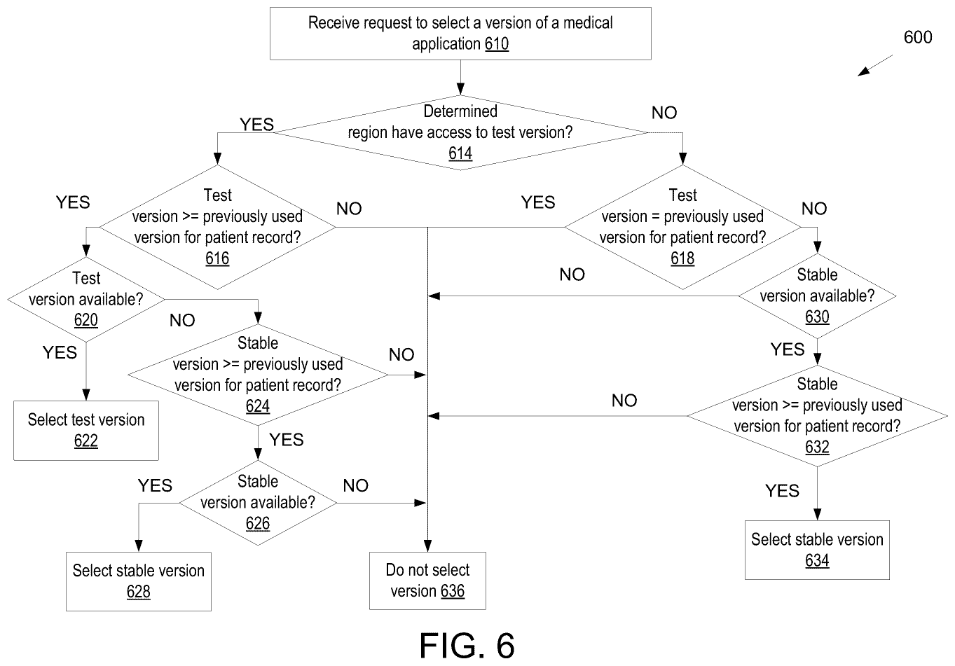

FIG. 6 illustrates a flow diagram for a method 600 of selecting a version of a medical application for execution based at least in part on region, in accordance with an embodiment. A user base and/or computing devices that use a medical application may be divided into regions (e.g., geographic regions or other groups). Regions may be based on geography, political boundaries, doctors, and so on. In embodiments, regions may be based on any set of features, and may be used to divide treatment cases into groups. In embodiments, an administrator may specify whether particular regions can run a test version of the medical application. Additionally, an administrator may specify a percentage of cases in a region to be processed using the test version of the medical application and/or a percentage of users of the medical application in a region that may use the test version of the medical application. In an example, different countries may have different regulations of medical software. Accordingly, a version of the medical application may be allowed for use under particular circumstances in one country, but may not be allowed for user under those particular circumstances in another country.

At block 610 of method 600, processing logic receives a request to select a version of a medical application. The request may include an indication of a region at which the medical application is to be used. The request may also include an identifier of a patient record to be accessed by the medical application. The request may also include a list of available versions of the medical application on a computing device that is to execute the medical application. At block 614, processing logic determines whether the region associated with the request has access to the test version of the medical application. If the region does have access to the test version of the medical application, the method continues to block 616. If the region does not have access to the test version of the medical application, the method continues to block 618.

At block 616, processing logic determines whether the test version was previously used for the medical record or if the test version has a higher version number than a version that was previously used for the medical record. If either condition is true, the method continues to block 620. If neither condition is true, the method continues to block 636.

At block 620, processing logic determines whether the test version of the medical application is available on the computing device that is to execute the medical application. If the test version is available, the method continues to block 622 and the test version is selected for execution. If the test version is not available, the method continues to block 624.

At block 624, processing logic determines whether a stable version of the medical application was previously used for the medical record or if the stable version of the medical application has a greater version number than the version of the medical application that was previously used for the medical record. If either condition is true, then the method continues to block 626. If neither condition is true, then the method continues to block 636.

At block 626, processing logic determines whether the stable version of the medical application is available. If so, the method continues to block 628, and the stable version of the medical application is selected. If the stable version is not available, the method continues to block 636.

At block 618, processing logic determines whether the version of the medical application that was previously used for the patient record is the test version of the medical application. If so, the method proceeds to block 636. Otherwise the method continues to block 630.

At block 630, processing logic determines whether the stable version of the medical application is available on the computing device that is to execute the medical application. If the stable version is not available, the method continues to block 636. If the stable version is available, the method proceeds to block 632.

At block 632, processing logic determines whether the stable version of the medical application was the version of the medical application that was previously used for the patient record or if the stable version of the medical application has a greater version number than the version of the medical application that was previously used for the patient record. If either condition is true, the method continues to block 634 and the stable version of the medical application is selected for execution. If neither condition is true, the method proceeds to block 636.

At block 636, processing logic determines that no version of the medical application should be selected, and that the medical application should not be executed.

FIG. 7 illustrates a flow diagram for a method 700 of selecting versions dependent medical applications for execution, in accordance with an embodiment. Method 700 is discussed with reference to multiple interdependent treatment planning applications. However, it should be understood that the same concepts discussed with reference to interdependent treatment planning applications also apply to other types of interdependent medical applications. According to method 800, at action 1 an administrator 720 may set parameters (e.g., one or more selection criteria) that define decision logic used to select versions of one or more medical applications. The administrator 720 may additionally update one or more selection criteria at any time. Setting and updating of the selection criteria may be performed in embodiments without redeployment or reinstallation of any of the versions of the medical applications, of the version selector 116, or of the launchers 730, 732, 734 for the medical applications in embodiments. In embodiments, the selection criteria may be used to limit a number of users that use a test or canary version of a medical application. A small subset of users may use the test version of the medical application, while a majority of users may continue to use an older stable version of the medical application. As the administrator gains more confidence in the new test version of the medical application, he or she may reduce restrictions on use of the test version and thus open it up for use by more users. For example, the administrator 720 may initially limit the test version of the medical application to one or a small number of regions or groups, and may over time add more and more regions or groups to the list of regions or groups that may use the test version of the medical application. Additionally, the administrator may initially limit the test version of the medical application to one or a small number of users, and may over time add more and more users to the list of users that may use the test version of the medical application.

At actions 2, 3, 4, one or more users 722 may attempt to open a particular medical application. For example, at action 2 a user 722 may attempt to open a first treatment planning application. At action 3 a user may attempt to open a second treatment planning application. At action 4, a user may attempt to open a third treatment planning application. Each of these treatment planning applications may be interrelated and/or interdependent. A first treatment planning application may operate on a patient case to start a treatment plan, and may later close that patient case. Later, a second treatment planning application may operate on the patient case to continue the treatment plan. Later, a third treatment planning application may operate on the patient case to continue to treatment plan. In some embodiments, a first treatment planning application initiates launching of another treatment planning application on which it depends. For example, chains of medical applications may be launched in embodiments, where for each application in the chain a version may be selected based at least in part on the versions of one or more prior medical applications already launched in the chain. In one example, the first treatment planning application comprises a computer aided drafting (CAD) treatment planning application, the second treatment planning application comprises a treatment planning generation application, and the third treatment planning application comprises a treatment planning calculation application.

At each launch of a treatment planning application to open a patient record, an appropriate version of the treatment planning application may be determined. The appropriate version of the treatment planning application to launch may be based at least in part on which versions of other treatment planning applications have already opened the patient case. Additionally, some treatment planning applications may make calls to other treatment planning application or other applications on which the treatment planning application depends during execution of the treatment planning application. This may cause those other applications to launch. The other applications may have different versions, and it may be important to select appropriate versions of those other applications to launch based on which version of the treatment planning application is running.

At block 730, a first launcher (e.g., concierge application) for the first treatment planning application is executed, which queries version selector 116 for a determination of whether to execute a stable version 750 or a test version 780 of the first treatment planning application. The launcher 730 may send a query to version selector 116, which may notify launcher 730 of a version of the first treatment planning application to launch (e.g., whether to launch the stable version 750 or test version 780 of the first treatment planning application).

At block 732, a second launcher (e.g., concierge application) for the second treatment planning application is executed, which queries version selector 116 for a determination of whether to execute a stable version 755 or a test version 785 of the second treatment planning application. The launcher 732 may send a query to version selector 116, which may notify launcher 732 of a version of the second treatment planning application to launch (e.g., whether to launch the stable version 755 or test version 785 of the second treatment planning application).

At block 734, a third launcher (e.g., concierge application) for the third treatment planning application is executed, which queries version selector 116 for a determination of whether to execute a stable version 760 or a test version 790 of the third treatment planning application. The launcher 734 may send a query to version selector 116, which may notify launcher 734 of a version of the third treatment planning application to launch (e.g., whether to launch the stable version 760 or test version 790 of the third treatment planning application).

Responsive to receipt of a query from a launcher 730, 732, 734, version selector 116 may determine one or more parameters or conditions associated with a current query, which may include which versions of a treatment planning application in quest are available on a computing device of the launcher 730, 732, 734, a user account associated with the query, a region or group associated with the query, a patient record associated with the query, whether the patient record was previously accessed by any version of the first treatment planning application, whether the patient record was previously accessed or is currently being accessed by any version of the second treatment planning application and/or the third treatment planning application, and/or other information.

As shown, the first, second and third treatment planning applications may be interdependent. In one embodiment, the stable versions 750, 755, 760 of each of the treatment planning applications are dependent on each other and form a stable version dependency group 740. In one embodiment, the test versions 780, 785, 790 of each of the treatment planning applications are dependent on each other and form a test version dependency group 742. Based on such groups 740, 742, version selector 166 may determine, responsive to receipt of a query to select a version of treatment planning application for a patient record, whether the patient record is being accessed or has been accessed by any version of one or more of the other treatment planning applications. If a stable version of one of the other treatment planning applications has been used to access, or is being used to access, the patient record, then a stable version may be selected for the treatment planning application at hand. If a test version of one of the other treatment planning applications has been used to access, or is being used to access, the patient record, then a test version may be selected for the treatment planning application at hand.

In some embodiments, after a particular version of a medical application (e.g., treatment planning application) is executed, that medical application may rely on one or more other medical applications and may initiate execution of those one or more other medical applications. This may invoke a launcher for each of the one or more other medical applications, which may then send a query to version selector 116 as to which version of the other medical application should be launched. In some instances there is a one to one correspondence between compatible versions of the different medical applications. Alternatively, one version of one medical application may be compatible with multiple versions of another medical application. Accordingly, version selector may determine a version of the first medical application that has been used to open a medical record (or that is currently being used), may determine which versions of a second medical application are compatible with the determined version of the first medical application, and may select a highest compatible version of the second medical application for execution in an embodiment.

After a test version of one or more of the treatment planning applications has been in use, administrator 720 may check the health and performance of the test version(s) of the treatment planning applications. If the test versions are operating successfully, then the administrator may at action 9 promote the test versions to stable versions of the treatment planning applications. In one embodiment, processing logic may automatically determine whether one or more promotion criteria are satisfied (e.g., no errors or fewer than a threshold number of errors were detected for the test version of the medical application, a threshold number of patient records were successfully processed using the test version of the medical application, etc.). Responsive to determining that the promotion criteria are satisfied, processing logic may automatically promote the test version of the medical application to a stable version, or may generate a notice for an administrator 720 recommending that the test version be promoted to a stable version of the medical application. Promotion of the test version may be implemented by updating one or more selection criteria in embodiments.

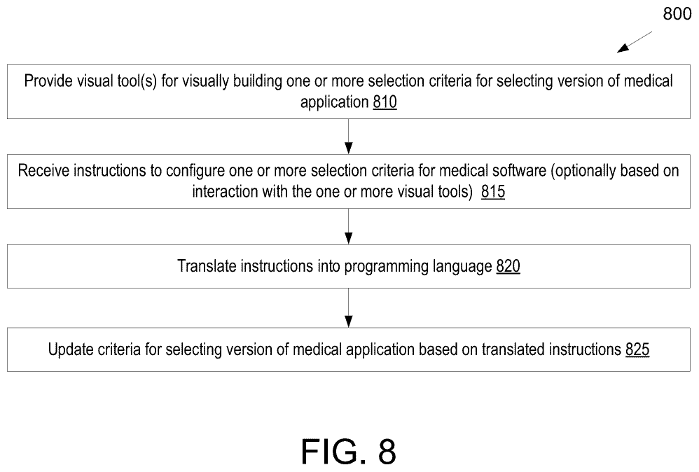

FIG. 8 illustrates a flow diagram for a method 800 of setting criteria for determining which version of multiple medical applications to execute, in accordance with an embodiment. At block 810 of method 800, processing logic provides one or more visual tools for visually building one or more selection criteria or rules for selecting a version of a medical application for execution. At block 815, processing logic receives instructions to configure one or more selection criteria for a medical application. The instructions may be received via user interaction with the one or more visual tools (e.g., via a user dragging and dropping blocks within a graphical user interface, where each block may represent an aspect of a rule for version selection). At block 820, processing logic may translate the received instructions, which may be received in a form other than the syntax of a programming language, into the syntax of a programming language. At block 825, processing logic may update criteria for selecting a version of the medical application based on the translated instructions. The update to the selection criteria may be made without reinstalling or redeploying any applications. For example, the update may be implemented merely up updating a configuration file for a selection application or service.

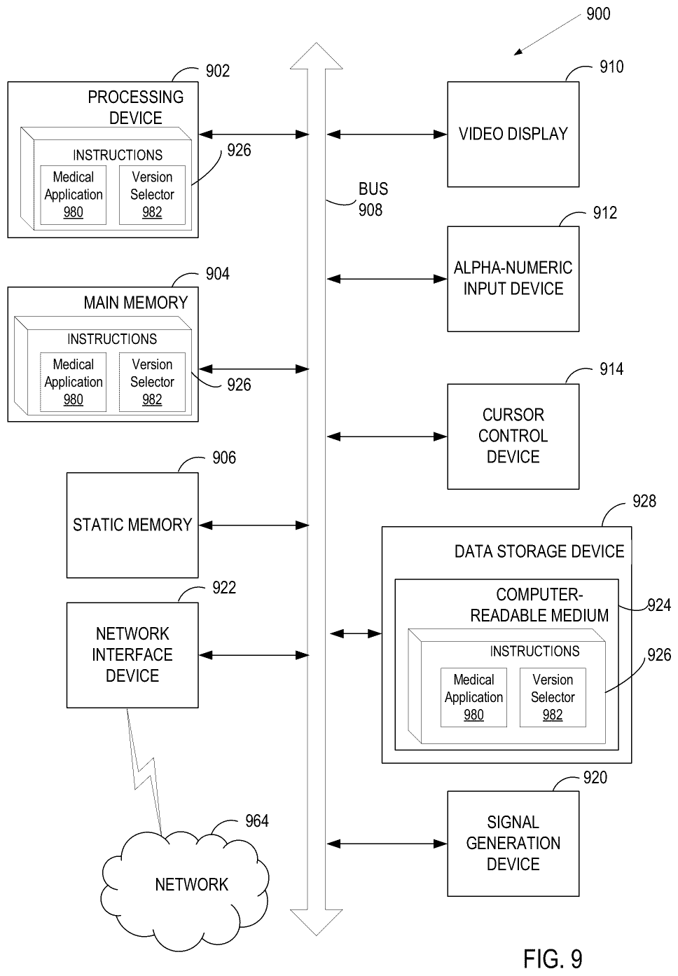

FIG. 9 illustrates a diagrammatic representation of a machine in the example form of a computing device 900 within which a set of instructions, for causing the machine to perform any one or more of the methodologies discussed herein, may be executed. In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a personal digital assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computer device 900 corresponds to computing device 105 and/or computing device 106 of FIG. 1.

The example computing device 900 includes a processing device 902, a main memory 904 (e.g., read-only memory (ROM), flash memory, dynamic random access memory (DRAM) such as synchronous DRAM (SDRAM), etc.), a static memory 906 (e.g., flash memory, static random access memory (SRAM), etc.), and a secondary memory (e.g., a data storage device 928), which communicate with each other via a bus 908.

Processing device 902 represents one or more general-purpose processors such as a microprocessor, central processing unit, or the like. More particularly, the processing device 902 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 902 may also be one or more special-purpose processing devices such as an application specific integrated circuit (ASIC), a field programmable gate array (FPGA), a digital signal processor (DSP), network processor, or the like. Processing device 902 is configured to execute the processing logic (instructions 926) for performing operations and steps discussed herein.

The computing device 900 may further include a network interface device 922 for communicating with a network 964. The computing device 900 also may include a video display unit 910 (e.g., a liquid crystal display (LCD) or a cathode ray tube (CRT)), an alphanumeric input device 912 (e.g., a keyboard), a cursor control device 914 (e.g., a mouse), and a signal generation device 920 (e.g., a speaker).

The data storage device 928 may include a machine-readable storage medium (or more specifically a non-transitory computer-readable storage medium) 924 on which is stored one or more sets of instructions 926 embodying any one or more of the methodologies or functions described herein, such as instructions for multiple versions of a medical application 980, a launcher for medical application 980 and/or for a version selector 982 for selecting a version of the medical application 980. In embodiments, version selector 982 corresponds to version selector 116 and medical application corresponds to medical application 120 of FIG. 1A. A non-transitory storage medium refers to a storage medium other than a carrier wave. The instructions 926 may also reside, completely or at least partially, within the main memory 904 and/or within the processing device 902 during execution thereof by the computer device 900, the main memory 904 and the processing device 902 also constituting computer-readable storage media.

The computer-readable storage medium 924 may also be used to store versions of medical application 980, a launcher for medical application 980 and/or to store version selector 982. The computer readable storage medium 924 may also store a software library containing methods for the medical application 980, the launcher for the medical application and/or the version selector 982. While the computer-readable storage medium 924 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium other than a carrier wave that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A system comprising:
a computing device comprising a memory and one or more processors, the computing device configured to:
receive a command to execute a medical application having a plurality of versions, the plurality of versions comprising at least a first version and a second version;
determine whether one or more selection criteria associated with the second version of the medical application are satisfied; and
responsive to determining that the one or more selection criteria are satisfied, execute the second version of the medical application.

2. The system of claim 1, wherein a single version of the medical application is exposed to users.

3. The system of claim 1, wherein the computing device is further configured to:
responsive to determining that the one or more selection criteria are not satisfied, execute the first version of the medical application.

4. The system of claim 1, wherein the first version is a production version of the medical application, and wherein the second version is a test version of the medical application.

5. The system of claim 4, wherein the computing device is further configured to:
determine whether the test version of the medical application satisfies one or more promotion criteria; and
responsive to determining that the test version of the medical application satisfies the one or more promotion criteria, promote the test version of the medical software to a new production version of the medical application.

6. The system of claim 1, wherein the computing device is further configured to:
responsive to determining that each of the plurality of versions of the medical application are prohibited from use, output an error.

7. The system of claim 1, wherein the computing device is further configured to:
determine an additional medical application that depends on the medical application or on which the medical application depends, the additional medical application having a plurality of versions;
determine a version of the plurality of versions of the additional medical application that satisfies one or more additional selection criteria; and
execute the determined version of the additional medical application.

8. The system of claim 7, wherein determining the version of the plurality of versions of the additional medical application that satisfies the one or more additional selection criteria comprises:
determining that the version of the additional medical application is a highest version of the additional medical application that is associated with the second version of the medical application.

9. The system of claim 1, wherein the computing device is further configured to:
determine whether the second version of the medical application is on a blacklist, wherein an execution criterion of the one or more selection criteria is satisfied by the second version of the medical application not being on the blacklist.

10. The system of claim 1, wherein the computing device is further configured to:
determine a user account associated with the command to execute the medical application; and
determine whether the user account is authorized to execute the second version of the medical application, wherein an execution criterion of the one or more selection criteria is satisfied by the user account being authorized to execute the second version of the medical application.

11. The system of claim 1, wherein the computing device is further configured to:
determine a current geographic region; and
determine whether the second version of the medical application authorized for use in the current geographic region, wherein an execution criterion of the one or more selection criteria is satisfied by the second version of the medical application being authorized for use in the current geographic region.

12. The system of claim 1, wherein the computing device is further configured to:
determine a number of instances of the second version of the medical application being run; and
determine whether the number of instances of the second version of the medical application is below a threshold, wherein an execution criterion of the one or more selection criteria is satisfied by the number of instances of the second version of the medical application being below the threshold.

13. The system of claim 1, wherein the computing device is further configured to:
determine a patient record to be accessed by the medical application; and
determine whether the patient record was previously accessed using any version of the medical application, wherein a criterion of the one or more selection criteria is satisfied base on at least one of a) the patient record having been previously accessed by the second version of the medical application or b) the patient record not having been accessed by any other version of the medical application.

14. The system of claim 1, wherein the computing device is further configured to:
determine a number of problems associated with the second version of the medical application; and
determine whether the number of problems associated with the second version of the medical application is below a threshold, wherein an execution criterion of the one or more selection criteria is satisfied by the number of problems associated with the second version of the medical application being below the threshold.

15. The system of claim 1, wherein the computing device is further configured to:
determine a patient record to be accessed by the medical application;
determine one or more details of the patient record; and
determine which of the plurality of versions of the medical application to execute based at least in part on the one or more details of the patient record.

16. The system of claim 15, wherein the one or more details comprise at least one of a region identity, a doctor identity, a type of treatment, or a user account.

17. The system of claim 1, wherein the medical application comprises at least one of a restorative dental treatment planning application or an orthodontic treatment planning operation.

18. The system of claim 1, wherein the computing device is further configured to:
receive instructions to configure the one or more selection criteria;
translate the instructions into a programming language; and
update the one or more selection criteria based on the translated instructions.

19. The system of claim 1, wherein the computing device is further configured to:
update the one or more selection criteria without redeployment or reinstallation of any of the plurality of versions of the medical application.

20. A non-transitory computer readable medium comprising instructions that, when executed by a processing device, cause the processing device to perform operations comprising:
receiving a command to execute a medical application having a plurality of versions, the plurality of versions comprising at least a first version and a second version;
determining whether one or more selection criteria associated with the second version of the medical application are satisfied; and
responsive to determining that the one or more selection criteria are satisfied, executing the second version of the medical application.
